(19) United States
(12) Patent Application Publication
(10) Pub. No.: US 2007/0264237 A1
(43) Pub. Date: Nov. 15, 2007

(54) Use of Il-18 Binding Protein in Inflammations

(75) Inventors: Menachem Rubinstein, Rehovot (IL); Daniela Novick, Rehovot (IL); Vladimir Hurgin, Ashdod (IL)

Correspondence Address: MARSHALL, GERSTEIN & BORUN LLP, 233 S. WACKER DRIVE, SUITE 6300, SEARS TOWER, CHICAGO, IL, 60606, US

(73) Assignee: YEDA RESEARCH AND DEVELOPMENT CO. LTD., P.O. BOX 95 Rehovot Israel 76100 (IL)

(21) Appl. No.: 10/584,805

(22) PCT Filed: Dec. 27, 2004
(86) PCT No.: PCT/IL04/01170

§ 371 Date: Mar. 26, 2007

(30) Foreign Application Priority Data
Dec. 31, 2003 (IL) 159670

(51) Int. Cl. A61K 38/20; A61K 39/395; A61P 29/00; A61P 43/00; C07K 14/435; C07K 14/47; C07K 14/54; C07K 14/545; C07K 14/715; C07K 16/18; C07K 16/28

(52) U.S. Cl. 424/93.7; 424/93.1; 514/12; 514/44

(57) ABSTRACT

The invention relates to the combined use of an IL-1 antagonist/inhibitor and IL-18 binding protein in inflammatory diseases.

### FIELD OF THE INVENTION

The invention relates to the combined use of interleukin-l antagonist/inhibitor and interleukin-18 binding protein (IL-18BP) in inflammatory diseases, such as rheumatoid arthritis.

### BACKGROUND OF THE INVENTION

Autoimmune and inflammatory diseases are mediated by several pro-inflammatory cytokines including TNF, IL-1, IL-6, IL-12, IL-17, IL-18, IL-23 and interferon gamma (IFN-gamma). Blocking one or more of these cytokines may have a significant effect on the course and symptoms of these diseases.

Rheumatoid arthritis is an inflammatory arthritis in which joints, usually including those of the hands and feet, are inflamed, resulting in swelling, pain, and often the destruction of joints. Worldwide, rheumatoid arthritis develops in about 1% of the population, regardless of race or country of origin, affecting women 2 to 3 times more often than men. Usually, rheumatoid arthritis first appears between 25 and 50 years of age, but it may occur at any age. Rheumatoid arthritis can occur in children, the disease is then called juvenile rheumatoid arthritis, and the symptoms and prognosis are somewhat different.

Rheumatoid arthritis is considered an autoimmune disease. Components of the immune system attack the soft tissue that lines the joints and can also attack connective tissue in many other parts of the body, such as the blood vessels and lungs. Eventually, the cartilage, bone, and ligaments of the joint erode, causing deformity, instability, and scarring within the joint. The joints deteriorate at a highly variable rate.

Once autoreactive T cells have been “primed” by contact with a foreign antigen, they release several cytokines-including TNF, interleukin-1 (IL-1), and others-that stimulate other immune cells to attack joints.

Due to the central role of IL-1 in inflammation in general, and in RA in particular, efforts are currently invested in developing antagonistic drugs to IL-1.

IL-1 receptor antagonist (IL-1Ra) is a naturally occurring soluble form of an IL-1 molecule, which binds to both IL-1 receptors but induces no biological effects. As such, it antagonizes the natural activities of both IL-1 variants. IL-1Ra is believed to function as a natural regulator of IL-1 activity in vivo. For a review of IL-1 receptor antagonists and complement receptor antagonists see Mantovani et al. (18).

Kineret—anakinra is a recombinant version of the human Interleukin-1 receptor antagonist (IL-1Ra). Kineret is approved for the treatment of rheumatoid arthritis (RA). It acts as a competitive inhibitor of the pro-inflammatory cytokines IL-1 alpha and beta, which are released at inflammatory sites by immune cells and by local tissue cells. Kineret was approved for the treatment of rheumatoid arthritis based on clinical trials, which demonstrated that it prevents damage to cartilage and bones resulting from the inflammatory process.

IL-1 receptors have been cloned and are under development as antagonists of IL-1, i.e. soluble IL-1 receptor type 1 and 2, for possible treatment of rheumatoid arthritis, allergy, asthma, systemic lupus erythematosus (SLE), IBD, septic shock, osteoarthritis and other inflammatory disorders (Biotechnology eds Rehm, Reed, Puhler and Stadler volume 5a, p 150).

Interleukin-18 binding protein (IL-18BP) is a specific inhibitor of IL-18 (16), associated with arthritis (19) and with many other inflammatory and/or autoimmune diseases (20, 21, 22). IL-18BP reduced the severity of several experimental autoimmune diseases (19). Therefore, IL18BP is believed to function as a natural anti-inflammatory and immunosuppressive molecule neutralizing the effects of high IL18 levels during inflammation. IL-18BP is specifically induced by IFN-gamma as part of a negative feedback loop that regulates the induction of IFN-gamma by IL-18.

### SUMMARY OF THE INVENTION

The invention relates to the use of IL-18 binding protein (IL-18BP), or a mutein, functional derivative, fraction, circularly permuted derivative, fused protein, isoform and a salt thereof together with an IL-1 antagonist/inhibitor in the manufacture of a medicament for the treatment and/or prevention of an inflammatory disease such as allergy, asthma, systemic lupus erythematosus (SLE), IBD, septic shock, osteoarthritis and preferably rheumatoid arthritis.

More specifically, the antagonist/inhibitor of IL-1 is selected from caspase-1 (ICE) inhibitors, antibodies against IL-1, antibodies against any of the IL-1 receptor subunits, inhibitors of the IL-1 signaling pathway, antagonists of IL-1 which compete with IL-1 and block the IL-1 receptor such as IL-1 receptor antagonist (IL-1Ra) and IL-1 binding proteins, or an isoform, mutein, fused protein, functional derivative, active fraction or circularly permutated derivative thereof, or an antisense mRNAs, soluble IL-1 receptors, and IL-1R antibody.

In a preferred embodiment of the invention, the IL-1 antagonist is IL-1 receptor antagonist (IL-1Ra) and more preferably Kineret.

In another embodiments of the invention, the IL-18BP is PEGylated, fused to all or part of an immunoglobulin, preferably to the constant region of an immunoglobulin, and wherein the fused protein is still capable of binding to IL-18. More specifically, the immunoglobulin may be of the IgG1 or IgG2 isotype.

In one aspect, the invention relates to the simultaneous, or sequential use of IL-18BP and the IL-1 antagonist/inhibitor.

In another aspect, the invention relates to the use of IL-18BP in an amount of about 0.0001 to 10 mg/kg of body weight, or about 0.01 to 5 mg/kg of body weight or about 0.1 to 3 mg/kg of body weight or about 1 to 2 mg/kg of body weight. Preferably it relates to the use of IL-18BP in an amount of about 0.1 to 1000 mg/kg of body weight or 1 to 100 mg/kg of body weight or about 10 to 50 mg/kg of body weight.

The IL-1 antagonist/inhibitor, according to the invention, is used in an amount selected from 0.0001 to 10 mg/kg or about 0.01 to 5 mg/kg or body weight, or about 0.01 to 5 mg/kg of body weight or about 0.1 to 3 mg/kg of body weight or about 0.5 to 2 mg/kg of body weight or about 1 mg/kg of body weight, and preferably at about 1 mg/kg of body weight.

According to one embodiment of the invention, IL-18BP and/or IL-1 antagonist/inhibitor, may be used for subcutaneous administration, for intramuscular administration, daily, three times per week and/or once a week.

The invention provides the use of an IL-1 antagonist/inhibitor or an expression vector comprising the coding sequence of IL-1 antagonist/inhibitor and IL-18BP or an expression vector comprising the coding sequence of IL-18BP in the manufacture of a medicament for treatment and/or prevention of an inflammatory disease such as allergy, asthma, systemic lupus erythematosus (SLE), IBD, septic shock, osteoarthritis and preferably rheumatoid arthritis. Such medicament can be used, for example for gene therapy.

In addition, the invention provides the use of an IL-1 antagonist/inhibitor or a vector for inducing or enhancing the endogenous production of an IL-1 antagonist/inhibitor and IL-18BP or a vector for inducing or enhancing the endogenous production of IL-18BP in a cell in the manufacture of a medicament for the treatment and/or prevention of an inflammatory disease such as allergy, asthma, systemic lupus erythematosus (SLE), IBD, septic shock, osteoarthritis and preferably rheumatoid arthritis.

According to one embodiment of the invention, the use of an IL-1 antagonist/inhibitor or a cell that has been genetically modified to produce an IL-1 antagonist/inhibitor and IL-18BP or a cell that has been genetically modified to produce IL-18BP in the manufacture of a medicament for the treatment and/or prevention of an inflammatory disease such as allergy, asthma, systemic lupus erythematosus (SLE), IBD, septic shock, osteoarthritis and preferably rheumatoid arthritis, is provided.

In addition, the invention provides a pharmaceutical composition comprising a therapeutically effective amount of an antagonist/inhibitor of IL-1, preferably an IL-1Ra (such as Kineret), or a mutein, functional derivative, fraction, circularly permuted derivative, fused protein, isoform and a salt thereof and a therapeutically effective amount of IL-18BP or a mutein, functional derivative, fraction, circularly permuted derivative, fused protein, isoform and a salt thereof.

According to the invention, it is provided also a pharmaceutical composition comprising a therapeutically effective amount of an IL-1 antagonist/inhibitor or an expression vector comprising the coding sequence of IL-1 antagonist/inhibitor and IL-18BP or an expression vector comprising the coding sequence of IL-18BP.

The invention also provides a pharmaceutical composition comprising a therapeutically effective amount of an IL-1 antagonist/inhibitor or vector for inducing and/or enhancing the endogenous production of an IL-1 antagonist/inhibitor and IL-18BP or a vector for inducing and/or enhancing the endogenous production of IL-18BP in a cell.

In another embodiment, the invention provides a pharmaceutical composition comprising a therapeutically effective amount of an IL-1 antagonist/inhibitor or a cell that has been genetically modified to produce an IL-1 antagonist/inhibitor and IL-18BP or a cell that has been genetically modified to produce IL-18BP.

In addition, the invention provides a method of treatment and/or prevention of inflammatory disease such as allergy, asthma, systemic lupus erythematosus (SLE), IBD, septic shock, osteoarthritis and preferably rheumatoid arthritis, comprising administering to a host in need thereof an effective inhibiting amount of IL-18BP, or a mutein, functional derivative, fraction, circularly permuted derivative, fused protein, isoform or a salt thereof and an IL-1 antagonist/inhibitor or a mutein, functional derivative, fraction, circularly permuted derivative, fused protein, isoform and a salt thereof.

According to the invention, the antagonist/inhibitor of IL-1 may be for example, caspase-1 (ICE) inhibitors, antibodies against IL-1, antibodies against any of the IL-1 receptor subunits, inhibitors of the IL-1 signaling pathway, antagonists of IL-1 which compete with IL-1 and block the IL-1 receptor such as IL-1Ra and preferably Kineret, or an isoform, mutein, fused protein, functional derivative, active fraction or circularly permutated derivative thereof having essentially the same activity as the wild type IL-1 binding protein. Other IL-1 inhibitors can be e.g. IL-1 antisense mRNA, soluble IL-1 receptor, and IL-1R antibody.

According to the invention the IL-18BP in the method provided may be PEGylated and/or fused to another protein e.g. an immunoglobulin, preferably of the IgG1 isotype, or a fragment thereof such as e.g. the constant part of the immunoglobulin. In addition, the method of treatment of the invention contemplates simultaneous or sequential co-administration of IL-18BP and the IL-1 antagonist/inhibitor.

More specifically, the method of the invention provides IL-18BP administered in an amount of about 0.0001 to 10 mg/kg of body weight, or about 0.01 to 5 mg/kg of body weight or about 0.1 to 3 mg/kg of body weight or about 1 to 2 mg/kg of body weight. Also, IL-18BP may be administered in an amount of about 0.1 to 1000 mg/kg of body weight or 1 to 100 mg/kg of body weight or about 10 to 50 mg/kg of body weight. According to the method of the invention, IL-1 antagonist/inhibitor may be administered in an amount selected from 0.0001 to 10 mg/kg or about 0.01 to 5 mg/kg or body weight, or about 0.01 to 5 mg/kg of body weight or about 0.1 to 3 mg/kg of body weight or about 0.5 to 2 mg/kg of body weight or about 1 mg/kg of body weight, and preferably may be administered at about 1 mg/kg of body weight.

In one aspect of the invention, IL-18BP and/or IL-1 antagonist/inhibitor is/are administered subcutaneously and/or intramuscularly, daily, three times per week and/or once a week.

In addition, the invention provides a method of treatment and/or prevention of inflammatory disease such as allergy, asthma, systemic lupus erythematosus (SLE), IBD, septic shock, osteoarthritis and preferably rheumatoid arthritis, comprising administering to a host in need thereof an effective inhibiting amount an IL-1 antagonist/inhibitor or an expression vector comprising the coding sequence of IL-1 antagonist/inhibitor and IL-18BP or an expression vector comprising the coding sequence of IL-18BP. One of the methods according to the invention may be for example gene therapy.

In another embodiment, the invention provides a method of treatment and/or prevention of an inflammatory disease such as allergy, asthma, systemic lupus erythematosus (SLE), IBD, septic shock, osteoarthritis and preferably rheumatoid arthritis, comprising administering to a host in need thereof an effective inhibiting amount of an IL-1 antagonist/inhibitor or a vector for inducing and/or enhancing the endogenous production of an IL-1 antagonist/inhibitor and of an IL-18BP or a vector for inducing and/or enhancing the endogenous production of IL-18BP in a cell.

Also, the invention provides a method of treatment and/or prevention of an inflammatory disease such as allergy, asthma, systemic lupus erythematosus (SLE), IBD, septic shock, osteoarthritis and preferably rheumatoid arthritis, comprising administering to a host in need thereof an effective inhibiting amount of IL-1 antagonist/inhibitor or a cell that has been genetically modified to produce an IL-1 antagonist/inhibitor and IL-18BP or a cell that has been genetically modified to produce IL-18BP.

### BRIEF DESCRIPTION OF THE FIGURES

The invention is herein described, by way of example only, with reference to the accompanying figures. With specific reference now to the figures in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the figures making apparent to those skilled in the art how the several forms of the invention may be embodied in practice.

In the figures:

FIG. 1 shows the effect of IL-IRA on the antiviral activity of IFN-gamma Each column represents a twofold dilution series of IFN-gamma. The following reagents were added: Column 1, control; 2, antibodies to IL-1-beta; 3, IL-IRA 0.1 mg/ml; 4, IL-1RA 0.01 mg/ml; 5. IL-1 200 IU/ml +IL-1Ra 1 mg/ml; 6, IL-1 200 IU/ml+IL-1Ra 0.1 mg/ml; 7, IL-1 200 IU/ml +IL-1Ra 0.01 mg/ml; 8, IL-1 200 IU/ml.

FIG. 2 shows the effect of IL-1Ra on the level of several IFN-gamma-induced transcripts in human HaCat cells, as measured by semi-quantitative reverse-transcription PCR. Beta actin was used as a control.

FIG. 3 shows the effect of IL-1RA on the induction of IL-18BP by IFN-gamma in HaCat cells, as measured by ELISA of cell supernatants.

### DETAILED DESCRIPTION OF THE INVENTION

The invention relates to a combined use of IL-18 binding protein (IL-18BP), or a mutein, functional derivative, active fraction, circularly permuted derivative, fused protein, isoform and a salt thereof with an IL-1 antagonist/inhibitor in the manufacture of a medicament for the treatment and/or prevention of an inflammatory disease.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The invention is based on the unexpected finding that IL-1 is essential for induction of IL-18BP by interferon gamma. In light of such surprising results, the inventors conceived the necessity of supplementing IL-18BP to patients with inflammatory diseases receiving medication aimed to target inhibition of IL-1.

The inventors found that many of the immunoregulatory activities of IFN-gamma depend on the presence of IL-1. Thus, either IL-1 alpha and IL-1 beta must be present for efficient induction of IFN-gamma activities. The inventor found that blocking endogenous IL-1 with IL-1Ra or with antibodies to IL-1 results in a significant reduction of IFN-gamma-induced activities. Most of the activities of IFN-gamma are pro-inflammatory and their inhibition by IL-1Ra is beneficial for the patient. However, there is at least one activity of IFN-gamma, the induction of IL-18BP, which is anti-inflammatory. Indeed, the experimental data obtained show that IL-1Ra also blocked the induction of IL-18BP by IFN-gamma.

Hence, the invention relates to supplementing IL-1 antagonist/inhibitory therapy with IL-18BP in order to improve the efficacy of IL-1 antagonist/inhibitor as an anti-inflammatory agent.

Originally discovered as an antiviral agent (1), IFN-gamma has since been characterized as a cytokine with pleiotropic immunologic functions. IFN-gamma is primarily secreted by activated T cells and by natural killer (NK) cells, and can promote macrophage activation, mediate antiviral and antibacterial immunity, enhance antigen presentation, orchestrate activation of the innate immune system, coordinate lymphocyte-endothelium interaction, regulate Th1/Th2 balance towards Th1 cell-mediated responses, and control cellular proliferation and apoptosis (2,3). By and large the mechanism of IFN-gamma action is well established. Its cell-surface receptor, signal-transduction pathways and IFN-induced genes are well characterized (4, 5, 6).

The biological responses and genes induced by IFN-gamma are augmented by TNF and IL-1. Such synergy was reported for induction of e.g., inducible NO synthase (7), chemokines (8), the adhesion molecules ELAM-1 and ICAM-1 (9), IP-10 (10), TLR-2 and −4 (11) and class II MHC (12). Synergy was linked to cooperativity at the promoter level between GAS—the IFN-gamma-activated response element and the TNF/IL-1-activated NF-KB response element (13).

IL-1 is endogenously produced by many cell types and is either secreted to the medium (IL-1 beta), or is present on the cell surface (IL-1 alpha). In contrast, TNF is produced only by immune cells. As a result, many of the studies reporting biological activities of IFN-gamma were actually done in the presence of IL-1.

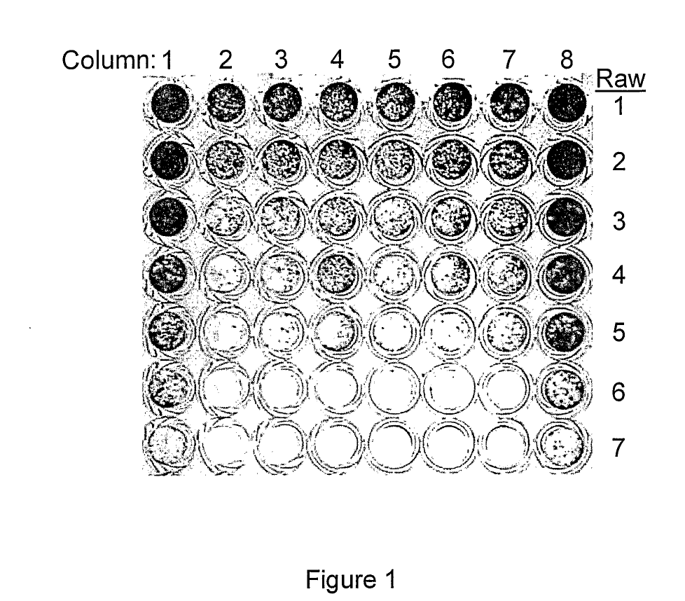

So far, no experimental data exist in which the biological activity of IFN-gamma was intentionally determined in the absence of IL-1. We have now measured several biological activities of IFN-gamma in the presence of either antibodies to IL-1 or the IL-1 receptor antagonist (IL-1Ra), which binds to the IL-1 receptor and blocks it from responding to IL-1. Unexpectedly, it was found that IL-1 is not only synergistically augmenting IFN-gamma actions, but is essential for many biological activities of IFN-gamma as well as for induction of genes by IFN-gamma. For example, when we measured the antiviral activity of IFN-gamma against vesicular stomatitis virus using human WISH cells, it was found that in the absence of IL-1 the antiviral potency of IFN-gamma was reduced by 90% (FIG. 1). Thus, in the absence of IL-1 activity, the specific activity of IFN-gamma was reduced by two orders of magnitude from 10⁷ IU/mg to 10⁵ IU/mg. This means that in the absence of IL-1, IFN-gamma is 1000 times less potent than IFN-alpha/beta (type I IFNs) whose specific activity is 10⁸ IU/mg. In contrast, no reduction in the antiviral activity of Type I IFNs was seen in the presence of IL-1Ra.

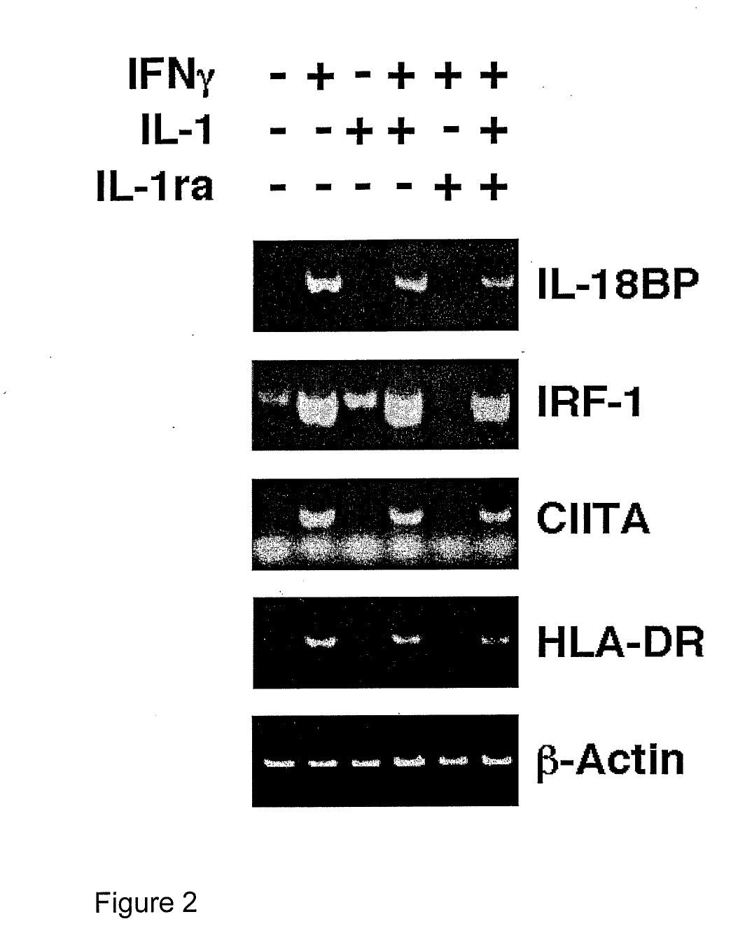

The effect of IL-1Ra on the ability of IFN-gamma to induce several genes, including IL-18BP, IRF-1, CIITA and HLA-DR was further investigated. For that purpose, a semi-quantitative RT-PCR using specific primers and comparing with beta-actin was employed. The results observed (FIG. 2) demonstrated that blocking IL-1 activity by IL-1Ra abrogated the induction of these genes by IFN-gamma.

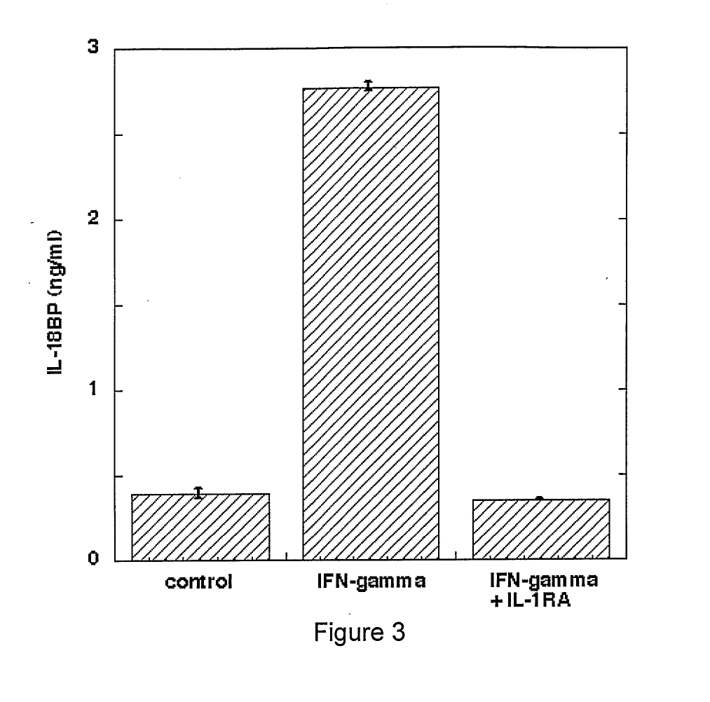

To further establish the dependency of IFN-gamma action on IL-1, the expression of IL-18BP by a specific ELISA (14) was measured. The result obtained confirmed those of the RT-PCR and induction of IL-18BP by IFN-gamma was blocked in the presence of IL-1Ra (FIG. 3).

Most of the activities of IFN-gamma are pro-inflammatory and their inhibition by IL-1Ra is beneficial for the patient. However, there is at least one activity of IFN-gamma—the induction of IL-18BP, which is anti-inflammatory. IL-1Ra has been approved for the treatment of rheumatoid arthritis (Kineret®). According the present invention IL-1Ra inhibits the induction of IL-18BP, whose only known specific inducer is IFN-gamma (15) . Such inhibition is problematic, as IL-18BP inhibits the activity of the pro-inflammatory cytokine IL-18 (16). Indeed, IL-18 is a major player in inflammatory and autoimmune diseases, including rheumatoid arthritis (17). Thus, inhibition of IL-18BP expression by administration of IL-1Ra is a disadvantage.

The present invention provides a combination therapy of IL-1 antagonist/inhibitor together with effective amounts of IL-18BP to overcome the disadvantage in using IL-1 antagonist/inhibitor such as IL-1Ra as a monotherapy in inflammatory and autoimmune diseases. Particularly, the invention relates to the combination therapy of an interleukin-1 antagonist/inhibitor such as IL-1Ra and IL-18BP in inflammatory diseases, such as RA.

The term “inhibitor of IL-1” within the context of this invention refers to any molecule modulating IL-1 production and/or action in such a way that IL-1 production and/or action is attenuated, reduced, or partially, substantially or completely prevented or blocked. The term “IL-1 antagonist/inhibitor” is meant to encompass inhibitors of IL-1 production as well as of inhibitors of IL-1 action.

An inhibitor of production can be any molecule negatively affecting the synthesis, processing or maturation of IL-1. The inhibitors considered according to the invention can be, for example, suppressors of gene expression of the interleukin IL-1, antisense mRNAs reducing or preventing the transcription of the IL-1 mRNA or leading to degradation of the mRNA, proteins impairing correct folding, or partially or substantially preventing secretion of IL-1, proteases degrading IL-1, once it has been synthesized, inhibitors of proteases cleaving pro-IL-1 in order to generate mature IL-1, such as inhibitors of caspase-1, and the like.

An inhibitor of IL-1 action can be an IL-1 antagonist, for example. Antagonists can either bind to or sequester the IL-1 molecule itself with sufficient affinity and specificity to partially or substantially neutralize the IL-1 or IL-1 binding site(s) responsible for IL-1 binding to its ligands (like, e.g. to its receptors). An antagonist may also inhibit the IL-1 signaling pathway, which is activated within the cells upon IL-1/receptor binding.

Inhibitors of IL-1 action may be also soluble IL-1 receptors or molecules mimicking the receptors, or agents blocking the IL-1 receptors, or IL-1 antibodies, such as polyclonal or monoclonal antibodies, or any other agent or molecule preventing the binding of IL-1 to its targets, thus diminishing or preventing triggering of the intra- or extracellular reactions mediated by IL-1.

An antagonist/inhibitor of IL-1 may be selected from caspase-1 (ICE) inhibitors, antibodies against IL-1, antibodies against any of the IL-1 receptor subunits, inhibitors of the IL-1 signaling pathway, antagonists of IL-1 which compete with IL-1 and block the IL-1 receptor, IL-1 receptor antagonist (IL-1Ra) and IL-1 binding proteins, or an isoform, mutein, fused protein, functional derivative, active fraction or circularly permutated derivatives thereof.

A preferred antagonist, according to the invention is IL-1Ra and a recombinant IL-1Ra such as Kineret®, or an isoform, mutein, fused protein, functional derivative, active fraction or circularly permutated derivative thereof.

The term “interleukin-18 binding protein” comprises also an IL-18BP mutein, functional derivative, fraction, circularly permuted derivative, fused protein, isoform and a salt thereof.

As used herein the term “muteins” refers to analogs of an IL-18BP, or analogs of a viral IL-18BP, in which one or more of the amino acid residues of a natural IL-18BP or viral IL-18BP are replaced by different amino acid residues, or are deleted, or one or more amino acid residues are added to the natural sequence of an IL-18BP, or a viral IL-18BP, without changing considerably the activity of the resulting products as compared with the wild type IL-18BP or viral IL-18BP. These muteins are prepared by known synthesis and/or by site-directed mutagenesis techniques, or any other known technique suitable therefore.

Any such mutein preferably has a sequence of amino acids sufficiently duplicative of that of an IL-18BP, or sufficiently duplicative of a viral IL-18BP, such as to have substantially similar activity to IL-18BP. One activity of IL-18BP is its capability of binding IL-18. As long as the mutein has substantial binding activity to IL-18, it can be used in the purification of IL-18, such as by means of affinity chromatography, and thus can be considered to have substantially similar activity to IL-18BP. Thus, it can be determined whether any given mutein has substantially the same activity as IL-18BP by means of routine experimentation comprising subjecting such a mutein, e.g., to a simple sandwich competition assay to determine whether or not it binds to an appropriately labeled IL-18, such as radioimmunoassay or ELISA assay.

Muteins of IL-18BP polypeptides or muteins of viral IL-18BPs, which can be used in accordance with the present invention, or nucleic acid coding therefore, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of IL-18BP polypeptides or proteins or viral IL-18BPs, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of IL18_BP polypeptides or proteins, or muteins of viral IL18_BPs, for use in the present invention include any known method steps, such as presented in U.S. Pat. Nos. RE 33,653, 4,959,314, 4,588,585 and 4,737,462, to Mark et al. 5,116,943 to Koths et al., 4,965,195 to Namen et al. 4,879,111 to Chong et al. and 5,017,691 to Lee et al; and lysine substituted proteins presented in U.S. Pat. No. 4,904,584 (Shaw et al).

The term “fused protein” refers to a polypeptide comprising an IL-18BP, or a viral IL-18 BP, or a mutein or fragment thereof, fused with another protein, which, e.g., has an extended residence time in body fluids. An IL-18BP or a viral IL-18BP may thus be fused to another protein, polypeptide or the like, e.g., an immunoglobulin or a fragment thereof.

“Functional derivatives” as used herein cover derivatives of IL-18BPs or a viral IL-18BP, and their muteins and fused proteins, which may be prepared from the functional groups which occur as side chains on the residues or the N- or C-terminal groups, by means known in the art, and are included in the invention as long as they remain pharmaceutically acceptable, i.e. they do-not destroy the activity of the protein which is substantially similar to the activity of IL-1 8BP, or viral IL-1 8BPs, and do not confer toxic properties on compositions containing it.

These derivatives may, for example, include polyethylene glycol side-chains, which may mask antigenic sites and extend the residence of an IL-18BP or a viral IL-18BP in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “fractions” of an IL-18BP, or a viral IL-18BP, muteins and fused proteins, the present invention covers any fragment or precursors of the polypeptide chain of the protein molecule alone or together with associated molecules or residues linked thereto, e.g., sugar or phosphate residues, or aggregates-of the protein molecule or the sugar residues by themselves, provided said fraction has substantially similar activity to IL-18BP.

The term “salts” herein refers to both salts of carboxyl groups and to acid addition salts of amino groups of the IL-18BP molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of IL-18BP, e.g. the ability to bind IL-18.

“Isoforms” of IL-18BP are proteins capable of binding IL-18 or fragment thereof, which may be produced by alternative splicing.

The term “circularly permuted derivatives” as used herein refers to a linear molecule in which the termini have been joined together, either directly or through a linker, to produce a circular molecule, and then the circular molecule is opened at another location to produce a new linear molecule with termini different from the termini in the original molecule. Circular permutations include those molecules whose structure is equivalent to a molecule that has been circularized and then opened. Thus, a circularly permuted molecule may be synthesized de novo as a linear molecule and never go through a circularization and opening step. The preparation of circularly permutated derivatives is described in WO95/27732.

These isoforms, muteins, fused proteins or functional derivatives retain the biological activity of IL-18BP, in particular the binding to IL-18, and preferably have essentially at least an activity similar to IL-18BP. Ideally, such proteins have a biological activity which is even increased in comparison to unmodified IL-18BP. Preferred active fractions have an activity which is better than the activity of IL-18BP, or which have further advantages, like a better stability or a lower toxicity or immunogenicity, or they are easier to produce in large quantities, or easier to purify.

The sequences of IL-18BP and its splice variants/isoforms can be taken from WO99/09063 or from Novick et al., 1999 (16), as well as from (24).

Functional derivatives of IL-18BP may be conjugated to polymers in order to improve the properties of the protein, such as the stability, half-life, bioavailability, tolerance by the human body, or immunogenicity. To achieve this goal, IL-18-BP may be linked e.g. to Polyethlyenglycol (PEG). PEGylation may be carried out by known methods, described in WO 92/13095, for example.

Therefore, in a preferred embodiment of the present invention, IL-18BP is PEGylated. In a further preferred embodiment of the invention, IL-18BP is a fused protein comprising all or part of an IL-18BP, which is fused to all or part of an immunoglobulin. The person skilled in the art will understand that the resulting fusion protein retains the biological activity of IL-18BP, in particular the binding to IL-18. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the IL-18BP sequence and the immunoglobulin sequence. The resulting fusion protein has improved properties, such as an extended residence time in body fluids (half-life), increased specific activity, increased expression level, or the purification of the fusion protein is facilitated.

In a preferred embodiment, IL-18BP is fused to the constant region of an Ig molecule. Preferably, it is fused to heavy chain regions, like the CH2 and CH3 domains of human IgGI, for example. The generation of specific fusion proteins comprising IL-18BP and a portion of an immunoglobulin are described in example 11 of WP99/09063, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

The IL-1 antagonist/inhibitor can be used simultaneously, sequentially or separately with IL-18BP in inflammatory diseases, such as RA. Advantageously, a combination of IL-18BP and an IL-1 inhibitor preferably IL-1Ra and more preferably Kineret® in the manufacture of a medicament for a patient suffering of RA can be used. The combination of the IL-1 antagonist/inhibitor and IL-18BP is suitable for the for the treatment and/or prevention of arthritis, in particular rheumatoid arthritis. The active components may be used simultaneously, sequentially, or separately.

In a preferred embodiment of the present invention, the IL-18BP is used in an amount of about 0.0001 to 10 mg/kg of body weight, or about 0.01 to 5 mg/kg of body weight or about 0.1 to 3 mg/kg of body weight or about 1 to 2 mg/kg of body weight. In yet a further preferred embodiment, the inhibitor of IL-18 is used in an amount of about 0.1 to 1000 mg/kg of body weight or 1 to 100 mg/kg of body weight or about 10 to 50 mg/kg of body weight.

The invention further relates to the combination of an IL-1 antagonist/inhibitor or an expression vector comprising the coding sequence of an IL-1 antagonist/inhibitor with IL-18BP or an expression vector comprising the coding sequence of IL-18BP in the preparation of a medicament for the prevention and/or treatment of arthritic conditions or arthritis, in particular rheumatoid arthritis and for the treatment of inflammatory disease. A gene therapeutical approach is thus used for treating and/or preventing the disease. Advantageously, the expression of the IL-18BP and/or IL-1 antagonist/inhibitor will then be in situ, thus efficiently blocking IL-18 directly in the tissue(s) and/or IL-1 or cells affected by the disease.

In order to treat and/or prevent inflammation, and preferably arthritis, the gene therapy vector comprising the sequence of IL-18BP and/or IL-1 antagonist/inhibitor may be injected directly into the diseased tissue e.g. diseased joint, thus avoiding problems involved in systemic administration of gene therapy vectors, like dilution of the vectors, reaching and targeting of the target cells or tissues, and of side effects.

The combined use of an IL-1 antagonist/inhibitor or a vector inducing or enhancing endogenous IL-1 antagonist/inhibitor with IL-18BP or a vector for inducing and/or enhancing the endogenous production of IL-18 BP in a cell normally silent for expression of IL-18BP, or which expresses amounts of IL-1 antagonist/inhibitor and/or IL-18BP which are not sufficient respectively, are also contemplated according to the invention. The vector may comprise regulatory sequences functional in the cells desired to express the IL-1 antagonist/inhibitor and/or IL-18BP. Such regulatory sequences may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

The invention further relates to the combined use of an IL-1 antagonist/inhibitor or of a cell that has been genetically modified to produce IL-1 antagonist/inhibitor and IL-18BP or of a cell that has been genetically modified to produce an inhibitor of IL-18 in the manufacture of a medicament for the treatment and/or prevention of an inflammatory disease such as rheumatoid arthritis.

The invention further relates to pharmaceutical compositions, particularly useful for prevention and/or treatment of inflammatory diseases, e.g. those selected from rheumatoid arthritis, allergy, asthma, systemic lupus erythematosus (SLE), IBD, septic shock, and osteoarthritis, which comprise a therapeutically effective amount of IL-18BP and a therapeutically effective amount of an IL-1 antagonist/inhibitor such as IL-1Ra and preferably Kineret®. As inhibitor of IL-1, the composition may comprise caspase-1 inhibitors, antibodies against IL-1, antibodies against any of the IL-1 receptor subunits, inhibitors of the IL-1 signaling pathway, antagonists of IL-1 which compete with IL-1 and block the IL-1 receptor, and IL-1 binding proteins, isoforms, muteins, fused proteins, functional derivatives, active fractions or circularly permutated derivatives thereof having the same activity.

IL-18BP and its isoforms, muteins, fused proteins, functional derivatives, active fractions or circularly permutated derivatives as described above and inhibitor of IL-1 such as caspase-1 inhibitors, antibodies against IL-1, antibodies against any of the IL-1 receptor subunits, inhibitors of the IL-1 signaling pathway, antagonists of IL-1 which compete with IL-1 and block the IL-1 receptor, il-I receptor antagonist, and IL-1 binding proteins or isoforms, muteins, fused proteins, functional derivatives, active fractions or circularly permutated derivatives thereof having similar or enhanced IL-18 and IL-1 activity than the wild type molecules respectively are the preferred active ingredients of the pharmaceutical compositions.

The IL-1 antagonist/inhibitor comprised in the pharmaceutical composition is preferably IL-1Ra, more preferably Kineret®.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amounts of the active proteins will be a function of many variables, including the type of antagonist, the affinity of the antagonist for IL-1, any residual cytotoxic activity exhibited by the antagonists, the route of administration, the clinical condition of the patient (including the desirability of maintaining a non-toxic level of endogenous IL-18 activity).

A “therapeutically effective amount” is such that when administered, the IL-18BP results in inhibition of the biological activity of IL-18 and the IL-1 antagonist/inhibitor results in inhibition of the biological activity of IL-1.

The dosage administered of IL-18BP and IL-1 antagonist/inhibitor, each as single or multiple doses, to an individual will vary depending upon a variety of factors, including IL-18 BP and IL-1 antagonist/inhibitor pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of IL-18 in an individual.

According to the invention, the IL-18BP is used in an amount of about 0.0001 to 10 mg/kg or about 0.01 to 5 mg/kg or body weight, or about 0.01 to 5 mg/kg of body weight or about 0.1 to 3 mg/kg of body weight or about 1 to 2 mg/kg of body weight. Further preferred amounts of the IL-18 inhibitors are amounts of about 0.1 to 1000 mg/kg of body weight or about 1 to 100 mg/kg of body weight or about 10 to 50 mg/kg of body weight. The IL-1 antagonist, is used in an amount of about 0.0001 to 10 mg/kg or about 0.01 to 5 mg/kg or body weight, or about 0.01 to 5 mg/kg of body weight or about 0.1 to 3 mg/kg of body weight or about 0.5 to 2 mg/kg of body weight or preferably at about I mg/kg of body weight.

The route of IL-18BP and/or IL-1 antagonist/inhibitor administration, which is preferred according to the invention, is administration by subcutaneous route. Intramuscular administration is further preferred according to the invention.

In further preferred embodiments, the IL-18BP and/or IL-1 antagonist/inhibitor are administered daily, every other day, three times per week or once a week.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the IL-18 inhibitor can be administered prophylactically or therapeutically to an individual prior to, simultaneously or sequentially with IL-1 antagonist/inhibitor (e.g. multiple drug regimens), in a therapeutically effective amount. Active agents that are administered simultaneously with other therapeutic agents can be administered in the same or different compositions.

In addition the invention relates to method of treatment and/or prevention of inflammatory disease comprising administering to a host in need thereof an effective inhibiting amount of IL-18BP, or a mutein, functional derivative, fraction, circularly permuted derivative, fused protein, isoform and a salt thereof and an IL-1 antagonist/inhibitor.

The invention further relates to a method for the preparation of a pharmaceutical composition comprising admixing an effective amount of an IL-18 BP and IL-1 antagonist/inhibitor, preferably IL-1Ra with a pharmaceutically acceptable carrier.

Having now described the invention, it will be more readily understood by reference to the following examples that are provided by way of illustration and are not intended to be limiting of the present invention.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al., (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al., (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the -art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### IL-1Ra inhibits the antiviral activity of IFN-gamma

In order to explore whether IL-1 must be present for induction of IFN-gamma antiviral activities, the effect of blocking endogenous and/or exogenous IL-1, with IL-1Ra or with IL-1 specific antibodies, in the protective antiviral effects of interferon gamma was explored.

Human WISH cells (4×10⁴ cells/well; ATCC CCL-25) were seeded in 96-well plates in 0.1 ml DMEM +10% FBS. IFN-gamma (0.1 ml, 2000 IU/ml) was added to each well of the first (top) row and then serially twofold diluted. Various reagents were then added to each well of a given column as follows. Column 1, no additive; column 2 anti IL-1 beta antibodies (PeproTech Inc. Rocky Hill N.J., 0.5 microgram/ml); column 3, IL-1Ra 0.1 30 mg/ml; column 4 IL-1Ra 0.01 mg/ml; column 5 IL-1 beta 200 IU/ml and IL-1Ra 1 mg/ml; column 6 IL-1 beta 200 IU/ml and IL-1Ra 0.1 mg/ml; column 7 IL-1 beta 200 IU/ml and IL-1Ra 0.01 mg/ml; column 8 IL-1 beta 200 IU/ml. The cultures were incubated overnight at 37° C. and 5% CO₂. On the next day the cultures were challenged with vesicular stomatitis virus and after 20 h the plate was stained with crystal violet and the extent of cytopathic effect was microscopically evaluated (23).

As can be seen in FIG. 1, IFN-gamma alone gave 50% protection in well No 5, representing 1000 IU/ml. Addition of antibodies to IL-1 beta (column 2) or IL-1Ra (column 3) reduced the antiviral activity of IFN-gamma and the endpoint (50% protection from viral cytopathic effect) was already seen at well No 2. This corresponds to 125 IU/ml, or about 90% inactivation as compared with IFN-gamma alone. In contrast, addition of exogenous IL-1 beta (row 8) has increased the antiviral potency of IFN-gamma by about 3-fold. Thus, the antiviral activity of IFN-gamma greatly depends on the presence of endogenous or exogenous IL-1.

### Example 2

### IL-1Ra inhibits the induction of several transcripts by IFN-gamma

The effect of IL-1Ra on the ability of IFN-gamma to induce several genes, including IL-18BP, IRF-1, CIITA and HLA-DR was further investigated. For that purpose, a semi-quantitative RT-PCR using specific primers and comparing with beta-actin was employed.

Human WISH or human HaCat cells (106) were plated in MEM and DMEM, respectively, supplemented with 10% FBS (2 ml) in 6-well plates. The plates were incubated overnight at 37° C. and 5% CO₂. On the next day, the cells were washed and subsequent treatments were carried out in medium containing 2% FBS. Cells were treated with various combinations of IFN-gamma (100 IU/ml) , IL-1-beta (200 IU/ml), IL-1Ra (10 microgram/ml) and anti-human IL-1b 0.05 mg/ml, added 1 hour prior to IFN-gamma. After 6 hours cells were harvested and total RNA was extracted using TRI reagent (Sigma). cDNA was prepared from the RNA using random hexamers and SuperscriptII (Invitrogen™, Leek, The Netherlands) according to the manufacturer's instructions. Semi-quantitative PCR was performed with the following primers:

hIL-18BP,
5′ CACGTCGTCACTCTCCTGG
and
5′ CGACGTGACGCTGGACAAC;
hIRF-1,
5′ GACCCTGGCTAGAGATGCAG
and
5′ GAGCTGCTGAGTCCATCAG;
hCIITA,
5′ CTGAAGGATGTGGAAGACCTGGGAAAGC
and
5′ GTCCCCGATCTTGTTCTCACTC;
hHLA-DR,
5′ GAGTTTGATGCTCCAAGCCCTCTCCCA
and
5′ CAGAGGCCCCCTGCGTTCTGCTGCATT;
human beta actin
5′ GTGGGGCGCCCCAGGCACCA
and
5′ CTCCTTAATGTCACGCACGATTTC.

Amplifications were done by initial denaturation (92° C., 2 min), 23 cycles of denaturation (92° C., 45 sec.), annealing (62° C., 45 sec) and extension (72° C., 45 sec), and final extension (72° C., 10 min). The resulting PCR products were resolved by agarose (1%) gel electrophoresis.

As can be seen in FIG. 2, IL-1Ra completely blocked the induction of mRNA of IL-18BP, IRF-1, CITA and HLA-DR, all of which are established IFN-gamma-induced genes.

### Example 3

### IL-1Ra inhibits the induction of IL-18BP by IFN-gamma

To further establish the dependency of IFN-gamma action on IL-1, the expression of IL-18BP by a specific ELISA (14) was measured.

The effect of IL-1Ra on induction of IL-18BP by IFN-gamma was determined in HaCat cells. Cultures of HaCat cells were treated with IFN-gamma (100 IU/ml) in the presence or absence of IL-1Ra as described in Example 2. Culture supernatants were collected after 48 hours and the concentration of IL-18BP was measured by a double antibody ELISA as described (14). Briefly, monoclonal antibody No 582.10 was used for capture of IL-18BP and a rabbit antiserum to human IL-18BP was used for detection. A preparation of recombinant human IL-18BP, provided by Serono Pharmaceutical Research Institute (Geneva, Switzerland), was used as a standard. As can be seen in FIG. 3, the level of IL-18BP in culture supernatants of control cultures was 0.39±0.03 ng/ml. Upon induction with IFN-gamma the level rose to 2.77±0.024 ng/ml, where as no induction by IFN-gamma was obtained in the presence of IL-1Ra (0.35±0.003 ng/ml).

The result obtained confirmed those of the RT-PCR and induction of IL-18BP by IFN-gamma was blocked in the presence of IL-1Ra.

### Example 4

### Coadministration of IL-1Ra and IL-18BP in rheumatoid arthritis patients Kineret® is supplied in single-use preservative free, 1 ml prefilled glass syringes with 27 gauge needles. Each prefilled glass syringe contains 0.67 ml (100 mg) of anakinra/Kineret. Kineret® is dispensed in packs containing 7 syringes (NDC 55513-177-07). It is also available in a 4×7 syringe dispensing pack containing 28 syringes (NDC 55513-177-28).

Rheumatoid arthritis adult patients treated with Kineret (daily administration of 100 mg) are further administered with IL-18BP. The American College of Rheumatology (ACR) responses are measured in such patients before, during and after the initiation of IL-18BP treatment.

Rheumatoid patients are administrated with both Kineret and IL-18BP by daily subcutaneous injections as follows: Kineret at 100 mg/day and IL-18BP at a dose within the range of 0.01 to 1000 mg/day.

Blood tests and/or synovial fluid examination, and/or x-ray examination of soft tissue are helpful to determine the improvement in patients receiving the combined treatment.

American College of Rheumatology (ACR) response criteria include changes in number of swollen joints, tender joints, physician global assessment of disease, patient global assessment of disease, patient assessment of pain, C-reactive protein, erythrocyte sedimentation rate, and health assessment questionnaire score.

It is expected that in patients with the combined treatment comprising IL-18BP and Kineret will have better ACR responses than in patients treated with Kineret alone.

ACR20 response requires a patient have a 20% reduction in the number of swollen and tender joints, and reduction of 20% in three of the following five indices; physician global assessment of disease, patient global assessment of disease, pain, C-reactive protein, erythrocyte sedimentation rate and health assessment questionnaire score.

ACR50 response requires a patient to have a 50% reduction in the number of swollen and tender joints, and reduction of 50% in three of the following five indices: physician global assessment of disease, patient global assessment of disease, pain, C-reactive protein, erythrocyte sedimentation rate and health assessment questionnaire score.

ACR70 response requires a patient have a 70% reduction in the number of swollen and tender joints, and a reduction of 70% in three of the following indices: physician global assessment of disease, patient global assessment of disease, pain, C-reactive protein, erythrocyte sedimentation rate and health assessment questionnaire score.

### Example 5

### Levels of serum IL-18BPa and IL-18 in RA patients before and after treatment with IL-1Ra.

Measurement of specific circulating cytokines and their natural inhibitors in health and disease provides information about their involvement in progression and severity of a disease. The level of IL-18 and its natural inhibitor, IL-18BP splice variant a, are monitored in RA patients and in RA patients treated with Kineret® (Daily doses of 100 mg) and compared to the levels found in healthy non-treated subjects by using specific ELISAs (14).

A. Levels of IL-18 and IL-18BPa in healthy subjects.

The mean level of IL-18 in 107 healthy donors is 64±17 pg/ml (14). The levels of IL-18BPa are the range from 0.5 ng/ml to as high as 7 ng/ml, with an average of 2.15+0.15 ng/ml (14). Because both IL-18 and IL-18BPa are concomitantly present in the serum, some of the IL-18 may be present in a complex with IL-18BPa. The level of free IL-18 is calculated based on the average level of total IL-18 (2.15 ng/ml). Free IL-18 was determined according to the law of mass action. The calculation is based on the following parameters: the concentrations of total IL-18 as determined by the ECL assay; the concentration of total IL-18BPa as determined by the ELISA; a 1:1 stoichiometry in the complex of IL-18BPa and IL-18 and a dissociation constant (Kd) of 0.4 nM (16 and 24). In an equilibrium system L+R=LR where L represents IL-18 and R represents IL-18BP the following equations are applicable:

1. Kd=[LR]/[Lfree][Rfree]

2. Lfree=Ltotal-LR

3. Rfree=Rtotal−LR

By substituting: Ltotal=64±17 pg/ml (mean level), Rtotal=2.15±0.15 ng/ml (average) and Kd=0.4 nM, it was found that in healthy subjects about 51.2 pg/ml IL-18 (about 80% from total) is in its free form (14).

B. Levels of IL-18 and IL-18BPa in RA patients.

The levels of IL-18 and of IL-18BPa are tested in sera samples from 60 RA patients. The levels of both IL-18 and IL-18BPa may be significantly more elevated in RA patients in comparison with the healthy subjects, and a broad distribution of the values may be observed. If no statistically significant correlation between creatinine levels and either IL-18 or IL-18BPa concentrations in these sera is observed (to be assessed by APACHE II score Knaus et al. 1993), this suggest that elevated IL-18 and IL-18BPa levels in these patients are not due to renal failure.

In case that serum IL-18 and IL-18BPa levels in RA patients varies considerably, the levels of free serum IL-18 in individual samples can be calculated. The calculations are done as previously described, using the same three equations and substitution of the Kd, Ltotal, Rtotal values found experimentally (14). The calculations may show that IL-18BPa reduces the level of free IL-18 in most RA patients. This effect may be particularly strong when total IL-18 is very high. Therefore, in such case most of the serum IL-18 is blocked by the circulating IL-18BPa.

C. Levels of IL-18 and IL-18BPa in RA patients treated with IL-1Ra. The levels of IL-18 and of IL-18BPa are tested in sera samples from 60 RA patients before and after treatment with IL-1Ra. The levels of IL-18BPa may be significantly less elevated in RA patients treated with IL-1Ra in comparison with the levels of IL-18BPa RA patients before the treatment (in B). Using the same three equations and substitution of the Kd, Ltotal, Rtotal values found experimentally (14) the levels of free IL-18 can be calculated and they may show that the levels of free IL-18 are increased in patients treated with IL-1Ra. The calculations may also show that IL-18BPa is too low in the circulation in order to reduce the level of free IL-18 in most IL-1Ra RA treated patients.

Therefore, administration of exogenous IL-18BPa to RA patients treated with IL-1Ra is expected to further lower the free circulating IL-18 level, causing alleviation of the disease outcome.

### REFERENCES

1. Wheelock, E., Interferon-like virus-inhibitor induced in human leukocytes by phytohemagglutinin. Science, 1965. 149: p. 310-311.
2. Billiau, A., Interferon-gamma: biology and role in pathogenesis. Adv Immunol, 1996.62: p. 61-130.
3. Boehm, U., et al., Cellular responses to interferon-gamma. Annu Rev Immunol, 1997. 15: p. 749-95.
4. Tau, G. and P. Rothman, Biologic functions of the IFN-gamma receptors. Allergy, 1999. 54(12): p. 1233-51.
5. Patrone, L., et al., Genes expressed during the IFN gamma-induced maturation of pre-B cells. Mol Immunol, 2002. 38(8): p. 597-606.
6. Rasschaert, J., et al., Global profiling of double stranded RNA- and IFN-gamma-induced genes in rat pancreatic beta cells. Diabetologia, 2003. 46(12): p. 1641-57.
7. Kwon, S. and S.C. George, Synergistic cytokine-induced nitric oxide production in human alveolar epithelial cells. Nitric Oxide, 1999.3(4): p. 348-57.
8. Visser, C. E., et al., Chemokines produced by mesothelial cells: huGRO-alpha, IP-10, MCP-1 and RANTES. Clin Exp Immunol, 1998.112(2): p. 270-5.
9. Doukas, J. and J. S. Pober, IFN-gamma enhances endothelial activation induced by tumor necrosis factor but not IL-1. J Immunol, 1990.145(6): p. 1727-33.
10. Ohmori, Y. and T. A. Hamilton, The interferon-stimulated response element and a kappa B site mediate synergistic induction of murine IP-10 gene transcription by IFN-gamma and TNF-alpha. J Immunol, 1995.154(10): p. 5235-44.
11. Faure, E., et al., Bacterial lipopolysaccharide and IFN-gamma induce Toll-like receptor 2 and Toll-like receptor 4 expression in human endothelial cells: role of NF-kappa B activation. J Immunol, 2001. 166(3): p. 2018-24.
12. Male, D. and G. Pryce, Synergy between interferons and monokines in MHC induction on brain endothelium. Immunol Lett, 1988. 17(3): p. 267-71.
13. Cheshire, J. L. and A. S. Baldwin, Jr., Synergistic activation of NF-kappaB by tumor necrosis factor alpha and gamma interferon via enhanced I kappaB alpha degradation and de novo IkappaBbeta degradation. Mol Cell Biol, 1997. 17(11): p. 6746-54.
14. Novick, D., et al., A novel IL-18BP ELISA shows elevated serum IL-18BP in sepsis and extensive decrease of free IL-18. Cytokine, 2001. 14(6): p. 334-42.
15. Hurgin, V., D. Novick, and M. Rubinstein, The promoter of IL-18 binding protein: activation by an IFN-gamma-induced complex of IFN regulatory factor 1 and CCAAT/enhancer binding protein beta. Proc Natl Acad Sci USA, 2002. 99(26): p. 16957-62.
16. Novick, D., et al., Interleukin-18 binding protein: a novel modulator of the Th1 cytokine response. Immunity, 1999. 10(1): p. 127-136.
17. Joosten, L. A., et al., Association of interleukin-18 expression with enhanced levels of both interleukin-1 beta and tumor necrosis factor alpha in knee synovial tissue of patients with rheumatoid arthritis. Arthritis Rheum, 2003. 48(2): p.339-47.
18. Mantovani A, Muzio M, Ghezzi P, Colotta F, Introna M. Negative regulators of the interleukin-1 system: receptor antagonists and a decoy receptor. Int J Clin Lab Res. 1996;26(1):7-14.
19. Plater-Zyberk C, Joosten L A, Helsen M M, Sattonnet-Roche P, Siegfried C, Alouani S, van De Loo F A, Graber P, Aloni S, Cirillo R, Lubberts E, Dinarello C A, van Den Berg W B, Chvatchko Y. Therapeutic effect of neutralizing endogenous IL-18 activity in the collagen-induced model of arthritis. J Clin Invest. 2001 December; 108(12):1825-32.
20. Nolsoe R L, Pociot F, Novick D, Rubinstein M, Kim S H, Dinarello C A, Mandrup-Poulsen T. Ann N Y Acad Sci. 2003 November; 1005:332-9. Mutation scan of a type 1 diabetes candidate gene: the human interleukin-18 binding protein gene.
21. Conti B, Jahng J W, Tinti C, Son J H, Joh T H. Induction of interferon-gamma inducing factor in the adrenal cortex. J Biol Chem. 1997 January 24;272(4):2035-7.
22. Pizarro T T, Michie M H, Bentz M, Woraratanadharm J, Smith M F Jr, Foley E, Moskaluk C A, Bickston S J, Cominelli F. IL-18, a novel immunoregulatory cytokine, is up-regulated in Crohn's disease: expression and localization in intestinal mucosal cells. J Immunol. 1999 June 1;162(11):6829-35.
23. Rubinstein S, Familletti P C, Pestka S. Convenient assay for interferons.

J Virol. 1981 February;37(2):755-8.
24. Kim S H, Eisenstein M, Reznikov L, Fantuzzi G, Novick D, Rubinstein M, Dinarello C A. Structural requirements of six naturally occurring isoforms of the IL-18 binding protein to inhibit IL-18. Proc Natl Acad Sci USA. 2000 February 1;97(3):1190-5.

1-30. (canceled)

31. A pharmaceutical composition comprising a therapeutically effective amount of anantagonist/inhibitor of IL-1, or a mutein, functional derivative, fraction, circularly permuted derivative, fused protein, isoform and a salt thereof and a therapeutically effective amount of IL-18BP or a mutein, functional derivative, fraction, circularly permuted derivative, fused protein, isoform and a salt thereof.

32. The pharmaceutical composition according to claim 31, wherein the antagonist/inhibitor of IL-1 is IL-1Ra.

33. The pharmaceutical composition according to claim 32, wherein the IL-1Ra is Kineret.

34. A pharmaceutical composition comprising a therapeutically effective amount of an IL-1 antagonist/inhibitor or an expression vector comprising the coding sequence of IL-1 antagonist/inhibitor and IL-18BP or an expression vector comprising the coding sequence of IL-18BP.

35. A pharmaceutical composition comprising a therapeutically effective amount of an IL-1 antagonist/inhibitor or vector for inducing and/or enhancing the endogenous production of an IL-1 antagonist/inhibitor and IL-18BP or a vector for inducing and/or enhancing the endogenous production of IL-18BP in a cell.

36. A pharmaceutical composition comprising a therapeutically effective amount of an IL-1 antagonist/inhibitor or a cell that has been genetically modified to produce an IL-1 antagonist/inhibitor and IL-18BP or a cell that has been genetically modified to produce IL-18BP.

37. A method of treatment and/or prevention of inflammatory disease comprising administering to a host in need thereof an effective inhibiting amount of IL-18BP, or a mutein, functional derivative, fraction, circularly permuted derivative, fused protein, isoform and a salt thereof and an IL-1 antagonist/inhibitor or a mutein, functional derivative, fraction, circularly permuted derivative, fused protein, isoform and a salt thereof.

38. The method according to claim 37, wherein the antagonist/inhibitor of IL-1 is selected from caspase-1 (ICE) inhibitors, antibodies against IL-1, antibodies against any of the IL-1 receptor subunits, inhibitors of the IL-1 signaling pathway, antagonists of IL- 1 which compete with IL-1 and block the IL-1 receptor, and IL-1 binding proteins, isoforms, muteins, fused proteins, functional derivatives, active fractions or circularly permutated derivatives thereof having essentially the same activity as an IL-1 binding protein.

39. The method according to claim 38, wherein IL-1 antagonist is IL-1Ra.

40. The method according to claim 39, wherein the IL-1Ra is Kineret.

41. The method according to claim 37, wherein the antagonist/inhibitor is selected from, antisense mRNAs, soluble IL-1 receptors, and IL-1R antibody.

42. The method according to any one of claims 37 to 41 or 67, wherein the IL-18BP is PEGylated.

43. The method according to any one of claims 37 to 41 or 67, wherein the inhibitor of IL-18 is a fused protein comprising all or part of an IL-18BP fused to all or part of an immunoglobulin, and wherein the fused protein binds to IL-18.

44. The method according to claim 43, wherein the fused protein comprises all or part of the constant region of an immunoglobulin.

45. The method according to claim 44, wherein the immunoglobulin is of the IgG1 or IgG2 isotype.

46. The method according to any one of claims 37 to 41 or 67, wherein IL-18BP and the IL-1 antagonist/inhibitor are administered simultaneously, or sequentially.

47. The method according to any one of claims 37 to 41 or 67, wherein IL-18BP is administered in an amount of about 0.0001 to 10 mg/kg of body weight, or about 0.01 to 5 mg/kg of body weight or about 0.1 to 3 mg/kg of body weight or about 1 to 2 mg/kg of body weight.

48. The method according to any one of claims 37 to 41 or 67, wherein IL-18BP is administered in an amount of about 0.1 to 1000 mg/kg of body weight or 1 to 100 mg/kg of body weight or about 10 to 50 mg/kg of body weight.

49. The method according to any one of claims 37 to 41 or 67, wherein the IL-1 antagonist/inhibitor is administered in an amount selected from 0.0001 to 10 mg/kg or about 0.01 to 5 mg/kg or body weight, or about 0.01 to 5 mg/kg of body weight or about 0.1 to 3 mg/kg of body weight or about 0.5 to 2 mg/kg of body weight or about 1 mg/kg of body weight.

50. The method according to claim 49, wherein the IL-1 antagonist/inhibitor is administered at about 1 mg/kg of body weight.

51. The method according to any one of claims 37 to 41 or 67, wherein IL-18BP is administered subcutaneously.

52. The method according to any one of claims 37 to 41 or 67, wherein IL-18BP is administered intramuscularly.

53. The method according to any one of claims 37 to 41 or 67, wherein the IL-1 antagonist/inhibitor is administered subcutaneously.

54. The method according to any one of claims 37 to 41 or 67, wherein the IL-1 antagonist/inhibitor is administered intramuscularly.

55. The method according to any one of claims 37 to 41 or 67, wherein IL-18BP is administered daily.

56. The method according to any one of claims 37 to 41 or 67, wherein IL-18BP is administered three times per week.

57. The method according to any one of claims 37 to to 41 or 67, wherein IL-18BP is administered once a week.

58. The method according to any one of claims 37 to 41 or 67, wherein the IL-1 antagonist/inhibitor is administered daily.

59. The method according to any one of claims 37 to to 41 or 67, wherein the IL-1 antagonist/inhibitor is administered three times per week.

60. The method according to any one of claims 37 to 41 or 67, the IL-1 antagonist/inhibitor is administered once a week.

61. A method of treatment and/or prevention of inflammatory disease comprising administering to a host in need thereof an effective inhibiting amount an IL- I antagonist/inhibitor or an expression vector comprising the coding sequence of IL-1 antagonist/inhibitor and IL-18BP or an expression vector comprising the coding sequence of IL-18BP.

62. The method of treatment and/or prevention according to claim 61 for gene therapy.

63. A method of treatment and/or prevention of an inflammatory disease comprising administering to a host in need thereof an effective inhibiting amount of an IL-1 antagonist/inhibitor or a vector for inducing and/or enhancing the endogenous production of an IL-1 antagonist/inhibitor and of an IL-18BP or a vector for inducing and/or enhancing the endogenous production of IL-18BP in a cell.

64. A method of treatment and/or prevention of an inflammatory disease comprising administering to a host in need thereof an effective inhibiting amount of IL-1 antagonist/inhibitor or a cell that has been genetically modified to produce an IL-1 antagonist/inhibitor and IL-18BP or a cell that has been genetically modified to produce IL-18BP.

65. The method according to any one of claims 61 to 64, wherein the inflammatory disease is selected from rheumatoid arthritis, allergy, asthma, systemic lupus erythematosus (SLE), IBD, septic shock, and osteoarthritis.

66. The method according to claim 65, wherein the inflammatory disease is rheumatoid arthritis.

67. The method according to claim 38, wherein the antagonist/inhibitor is selected from, antisense mRNAs, soluble IL-1 receptors, and IL-IR antibody.


SEQUENCE LISTING (12 sequences)

SEQ ID NO: 1 - protein, 3 aa, Artificial sequence
  Synthetic peptide
EFM

SEQ ID NO: 2 - protein, 13 aa, Artificial sequence
  Synthetic peptide
EFGAGLVLGGQFM

SEQ ID NO: 3 - DNA, 19 bp, Artificial sequence
  Synthetic primer
cacgtcgtcactctcctgg

SEQ ID NO: 4 - DNA, 19 bp, Artificial sequence
  Synthetic primer
cgacgtgacgctggacaac

SEQ ID NO: 5 - DNA, 20 bp, Artificial sequence
  Synthetic primer
gaccctggctagagatgcag

SEQ ID NO: 6 - DNA, 19 bp, Artificial sequence
  Synthetic primer
gagctgctgagtccatcag

SEQ ID NO: 7 - DNA, 28 bp, Artificial sequence
  Synthetic primer
ctgaaggatgtggaagacctgggaaagc

SEQ ID NO: 8 - DNA, 22 bp, Artificial sequence
  Synthetic primer
gtccccgatcttgttctcactc

SEQ ID NO: 9 - DNA, 27 bp, Artificial sequence
  Synthetic primer
gagtttgatgctccaagccctctccca

SEQ ID NO: 10 - DNA, 27 bp, Artificial sequence
  Synthetic primer
cagaggccccctgcgttctgctgcatt

SEQ ID NO: 11 - DNA, 20 bp, Artificial sequence
  Synthetic primer
gtggggcgccccaggcacca

SEQ ID NO: 12 - DNA, 24 bp, Artificial sequence
  Synthetic primer
ctccttaatgtcacgcacgatttc